(19) United States
(12) Patent Application Publication
(10) Pub. No.: US 2019/0142708 A1
(43) Pub. Date: May 16, 2019

(54) METHOD FOR PRODUCING TOPICAL COMPOSITION FOR HAIR-LOSS PREVENTION AND HAIR GROWTH

(71) Applicant: Ho Young YOON, Cheongju-si Chungcheongbuk-do (KR)

(72) Inventor: Ho Young YOON, Cheongju-si Chungcheongbuk-do (KR)

(21) Appl. No.: 16/099,444

(22) PCT Filed: Jul. 5, 2016
(86) PCT No.: PCT/KR2016/007244

§ 371 Date: Nov. 7, 2018

(51) Int. Cl. A61K 8/04; A61K 8/9789; A61K 8/73; A61Q 7/00

(52) U.S. Cl. CPC A61K 8/042; A61K 8/9789; A61K 2800/805; A61Q 7/00; A61K 8/732

(57) ABSTRACT

Disclosed is a method of producing a composition for preventing hair loss and promoting hair growth, which is capable of making the hear roots of the scalp to be smoothly contracted or relaxed, which is capable of enhancing the inherent physical properties of the hair roots and capable of stimulating the circulation of blood by discharging waste materials from the hair follicle, and which is convenient to use because the composition is not solidified. The composition of the present invention is produced by putting glutinous rice powders and corn flours into an Akebia Quinata base solution comprising an Akebia Quinata and a Selaginella, and then by mixing and heating them. The method according to the present invention comprises the steps of: (S1): putting an Akebia Quinata and a Selaginella into purified water and then heating the resulting solution so as to make an Akebia Quinata base solution; (S2): adding glutinous rice powders and corn flours to the Akebia Quinata base solution resulted from the step (S1), and then mixing and heating them so as to prepare a gel-like mixture; and (S3) cooling, filtering and sterilizing the gel-like mixture resulted from the step (S2).

### BACKGROUND OF THE INVENTION

### 1. Field of the Invention

The present invention is related to a method of producing a composition for preventing hair loss and promoting hair growth, and more particularly, to a method of producing a composition for preventing hair loss and promoting hair growth, which is capable of making the hear roots of the scalp to be smoothly contracted or relaxed, which is capable of enhancing the inherent physical properties of the hair roots and capable of stimulating the circulation of blood by discharging waste materials from the hair follicle, and which is convenient to use because the composition is not solidified. The composition of the present invention is produced by putting glutinous rice powders and corn flours into an Akebia Quinata base solution comprising an Akebia Quinata and a Selaginella, and then by mixing and heating them.

### 2. Description of the Prior Art

In general, the main causes of the baldness or the hair loss include the action of male hormones, the reduction of blood flow in the hair follicles, the hypersensitive scalp, the fat-soluble scalp, the stress, the genetic factors, and the aging, etc.

However, there has not yet been investigated and developed a definite cause and a clear treatment for the hair loss.

As one example of treating the alopecia, a minoxidil-containing medicine may be used. At first, the minoxidil-containing medicine was a therapeutic agent for hypertension developed for the purpose of lowering blood pressure. However, as a side effect of use it, a hair growth phenomenon appears and thereby it is now widely used as a hair growth solution. It was known that minoxidil-containing medicine may promote the hair growth by increasing the blood flow due to the effect of expanding blood vessels and by supplying nutrients to the hair follicles.

However, there is a serious drawback in the minoxidil-containing medicine. Since the minoxidil-containing medicine may increase the blood supply and stimulate the hair follicle, the effect of temporarily thickening the hair appears. However, if the use of the minoxidil-containing medicine is stopped, the effect is also stopped and returned to the original state.

As another example of treating the alopecia, a method of controlling the action of an antioxidant may be used as one method for regenerating hair follicles by enhancing immunity. However, this method does not regenerate hair in a fundamental form, but generate hair temporarily due to the cell division and the stimulation. Accordingly, the hair produced by this method has a short life span.

Therefore, many studies have been conducted to solve the above problems, and various hair growth agents for preventing and treating hair loss have been proposed. However, existing hair growth agents have improvement functions only for one kind of cause among various hair loss causes, and thus they do not have a great effect on hair growth.

For example, a technique related to hair growth agents for preventing and treating the hair loss is described in Korean Registered Patent No. 10-0982351 issued to SKBioland Co., Ltd., on Sep. 8, 2010, titled “Composition for promoting hair growth comprising magma seawater”. However, this technique requires to secure a sufficient amount of magma seawater and to perform the desalination process so as to produce mineral water. Accordingly, this leads to the economical waste. Also, it is very hard to carry out overall process.

Furthermore, a composition for promoting hair growth and a method of producing the same are described in Korean Laid-open Patent Publication No. 10-2011-0020691 in the name of Natural Beauty Co., Ltd., on Mar. 3, 2011, titled “Natural Herbal Composition For The Prevention of Depilation And Improvement of Hair Growth And Method For Preparing The Same”. This composition is mainly composed of herbal medicine extracts comprising Huttuynia cordata, Vervena offcinalis, natural henna, seaweed (kelp powder), etc. Furthermore, a composition for promoting hair growth and a method of producing the same are described in Korean Laid-open Patent Publication No. 10-2011-0056156 in the name of Natural Beauty Co., Ltd., on May 26, 2011, titled “A hair restorer comprising extract of rice straw and green perilla pod and Method for producing it”. This composition mainly comprises mixed extract of rice straw and green perilla pod. Since the constitutional elements of these patents are entirely different from those of the present invention, these patents are considered different from the present invention.

Furthermore, a composition for the hair comprising human body-friendly ingredients is described in Japanese Laid-open Patent Publication No. 2009-249361, in the name of General Cable Superconductors Limited, on Jul. 19, 2011, titled “Hair or scalp composition”. This composition mainly comprises extracts of liquorice root, ginko biloba leaf, citrus aurantium peel, ripened citrus reticulate peel, egg white, and a calcium compound. According to this Japanese Laid-open patent publication, it is required to break the egg white of the heated egg and then to immerse it in ethanol. Next, the resulting solution should be left for three weeks and then it should be squeezed by using a towel. Likewise, other raw materials must be squeezed through similar processes to that described above. In conclusion, the task of performing this technique is very complicated.

Furthermore, a composition for promoting hair growth and a method of producing the same are described in Korean Laid-open Patent Publication No. 10-2007-0107329, in the name of Young-Ki MIN, on Nov. 7, 2007, titled “Galenic composition for promoting hair-resotoration”. This composition comprises pine needles, wilfordi root, Korean angelica root, heukimja, crudus, thuja orientalis, rehmannia glutinosa, black soybean, green tea, kelp, sea mustard, spinach, which are separately pulverized and then powered by using separate processes. Accordingly, this leads to the economical waste. Also, it is very hard to carry out overall process.

In order to overcome the problems of the prior art as mentioned above, the present Applicant has developed a new composition. That is, a composition for preventing hair loss and promoting hair growth and a method for producing the same are described in Korean Registered Patent No. 10-1600968 issued to Ho-Yong YOUN, on Mar. 2, 2016, titled “Topical natural hair restorer composition and a method of manufacturing”. This composition used egg white of the heated egg as a protein source. However, the egg white of the heated egg was inconvenient to use because it can be solidified when it may be stored at room temperature or in the freezer. Further, according to this technique, since a relatively large amount of purified water is used, the manufacturing time becomes long. While implementing this technique, the present Applicant has found that improving blood circulation rather than feeding nutrients such as protein and the like is more effective in preventing hair loss and promoting hair growth.

Besides, other technologies mainly related to compositions for preventing hair loss and promoting hair growth have been disclosed in other patent documents—see Korean Registered Patent No. 10-0781470 issued to Baek-Ho YOUN, on Nov. 26, 2007, titled “Scalp-improving composition and scalp-improving cosmetic composition containing it”; Korean Laid-open Patent Publication No. 10-2008-0032127 in the name of KOHNO Kenji, on Apr. 14, 2008, titled “AGENT FOR HAIR GROWTH”; and Korean Laid-open Patent Publication No. 10-2011-0005363 in the name of Hankook Cosmetics Manufacturing Co., Ltd., on Jan. 18, 2011, titled “COMPOSITION FOR IMPROVING HAIR HAVING EFFECT OF DEPILATION PREVENTION AND GOOD HAIR”.

### SUMMARY OF THE INVENTION

Accordingly, the present invention has been made with a view to overcoming the foregoing problems of the prior arts. In particular, the present Applicant has further improved the technique disclosed in the above Korean Registered Patent No. 10-1600968 that was granted to the present Applicant. It is an object of the present invention provides a method of producing a composition for preventing hair loss and promoting hair growth, which is capable of making the hear roots of the scalp to be smoothly contracted or relaxed, which is capable of enhancing the inherent physical properties of the hair roots and capable of stimulating the circulation of blood by discharging waste materials from the hair follicle, and which is convenient to use because the composition is not solidified.

In accordance with the present invention, there is provided a method of producing a composition for preventing hair loss and promoting hair growth, the method comprising the following steps:

(S1): putting an Akebia Quinata and a Selaginella into purified water and then heating the resulting solution so as to make an Akebia Quinata base solution;

(S2): adding glutinous rice powders and corn flours to the Akebia Quinata base solution resulted from the step (S1), and then mixing and heating them so as to prepare a gel-like mixture; and

(S3) cooling, filtering and sterilizing the gel-like mixture resulted from the step (S2).

In the step (S1), the Akebia Quinata comprises at least one material selected from the group consisting of Akebia Trifoliata that is a stem of the Akebia Quinata, Akebia Trifoliata root that is a root of the Akebia Quinata, and Akebiae Fructus that is a fruit of the Akebia Quinata. The sum of the Akebia Quinata and the Selaginella to be used may be 90 to 150 g based on the purified water of 1,500˜2,000 cc.

In the step (S1), after putting the Akebia Quinata and the Selaginella into the purified water, the resulting solution is heated for 30 minutes at 100° C. and then heating process is stopped and then it is kept until the amount of the purified water is up to ⅔ of its original volume.

In the step (S2), the sum of the glutinous rice powder and the corn flour to be used may be 40 to 60 g based on the Akebia Quinata base solution of 900˜1,000 cc.

In the step (S3), the filtering process is performed by using a 300 mesh filter.

As described above, the composition according to the present invention is made of only natural substances and therefore it is harmless to human body. The composition can make the hear roots of the scalp to be smoothly contracted or relaxed and it can enhance the inherent physical properties of the hair roots and capable of stimulating the circulation of blood by discharging waste materials from the hair follicle. Since the composition does not use the egg white, the composition is not solidified and thereby resulting in convenience to use.

### BRIEF DESCRIPTION OF THE DRAWINGS

The above and other objects, features and advantages of the present invention will be more apparent from the following detailed description taken in conjunction with the accompanying drawings, in which:

FIGS. 1A and 1B are photographs taken before and after an experiment with a composition for preventing hair loss and promoting hair growth according to a preferred first embodiment of the present invention;

FIGS. 2A and 2B are photographs taken before and after an experiment with a composition for preventing hair loss and promoting hair growth according to a preferred second embodiment of the present invention;

FIGS. 3A and 3B are photographs taken before and after an experiment with a composition for preventing hair loss and promoting hair growth according to a preferred third embodiment of the present invention;

FIGS. 4A and 4B are photographs taken before and after an experiment with a composition for preventing hair loss and promoting hair growth according to a preferred fourth embodiment of the present invention;

FIGS. 5A and 5B are photographs taken before and after an experiment with a composition for preventing hair loss and promoting hair growth according to a preferred fifth embodiment of the present invention; and

FIGS. 6A and 6B are photographs taken before and after an experiment with a composition for preventing hair loss and promoting hair growth according to a preferred sixth embodiment of the present invention.

### DETAILED DESCRIPTION OF THE EXEMPLARY EMBODIMENTS

The drawings in the present application and their accompanying detailed description are directed to merely example embodiments of the invention. The advantages and other features of the methods disclosed herein will become more readily apparent to those having ordinary skill in the art from the following detailed description of certain preferred embodiments taken in conjunction with the drawings which set forth representative embodiments of the present invention.

At first, before describing the present invention in earnest, let us consider the raw materials to be used in the present invention.

Considering the growth cycle of hair (growth stage→catagen stage→resting stage→growth stage), alopecia may be caused by a long resting stage and a short growth stage. The present invention is based on the fact that when the resting stage is prolonged then the pores of the scalp may be clogged and thereby the hair growth becomes difficult. If the blood circulation in the scalp is improved, or the size of the skin's pore is enlarged, or the growth stage is prolonged by supplying the hair root with nutrients, then the vitality of the hair follicle can be activated. In this regard, the present invention intends to active the hair root by relaxing the pores of the skin and improving the blood circulation.

The term “natural substance” refers to a substance having natural ingredients with beneficial effects on the human body, which may be obtained by performing natural processes, such as boiling, slicing, drying, heating, extracting, and pulverizing, etc.

In the Korean Registered Patent No. 10-1600968 issued to the present applicant, the egg white of the heated egg was used as a protein source. The egg white refers to the egg of a bird or a reptile whose the egg yolk has been removed there from. Since the egg white is mainly composed of protein, the present applicant judged that it would be very effective in promoting hair growth. However, the egg white of the heated egg was inconvenient in use because it can be solidified when it may be stored at room temperature or in the freezer. Therefore, the egg white is no longer used in the present invention.

According to the present invention, glutinous rice powders and corn flours are used as raw materials. Glutinous rice powders and corn flours, which are natural substances, act to suppress lipid peroxidation of the scalp and to promote hair growth. According to the present invention, an Akebia Quinata is also used as raw materials. The Akebia Quinata has excellent effects on the anti-inflammatory action, the diuretic action and the emmenagogue action.

Since these raw materials used in the present invention can be also used as natural foods or medicines, there is no harm to human body and there is no toxicity or side effects during use or after use.

The composition for preventing hair loss and promoting hair growth according to the present invention was developed for the purpose of improving blood flow and blood circulation, strengthening hair follicles, activating hair follicles, and supplying nutrients. The natural ingredients employed in the present invention include ingredients that are essential for hair regeneration, and other ingredients that can improve the blood flow by promoting the contraction and relaxation of the scalp.

The effects and efficacy of such natural materials as described above are well known in the art, so a detailed description thereof will be omitted. Although general natural materials can be used in the present invention in consideration of the appropriate effect according to the present invention and other effects on the human body, it is preferable to select only domestic natural materials without having residual pesticides.

At first, in the present invention, glutinous rice is used as a main ingredient of the composition for preventing hair loss and promoting hair growth. Niacin and thiamine, which are extracted from glutinous rice and belong to the vitamin B group, are a major component of antioxidants, and vitamin E has the effect of preventing the aging of the scalp.

Next, in the present invention, corn flour is also used as a main ingredient of the composition for preventing hair loss and promoting hair growth. Corn flour has the effect of removing wastes from pores of the skin and improving the blood circulation to activate hair growth. Furthermore, because corn flour has an excellent diuretic action, it excretes waste and toxins from the human body. Corn flour contains important substances necessary for promoting hair growth such as Vitamin B1, B2 and Vitamin E. In detail, Vitamin B acts to inhibit the aging of the human body and Vitamin E acts to inhibit the generation of activated oxygen within the human body. Therefore, it is possible to remove wastes and sebum from the scalp, and it can suppress the lipid peroxidation due to the antibacterial action and thereby promoting hair growth.

Meanwhile, an Akebia Quinata base solution is prepared and used to produce the composition according to the present invention. Akebia Quinata is a good medicinal material for the kidneys and it has excellent anti-inflammatory and detoxifying effects on the hair growth area and promotes the blood circulation.

The Akebia Quinata belongs to deciduous shrubs of the Lardizabalaceae. The stem part of the Akebia Quinata is called as Akebia Trifoliata. The root of the Akebia Quinata is called as Akebia Trifoliata root and the fruit of the Akebia Quinata is called as Akebiae Fructus. The Akebia Trifoliata is effective in treating Phosphorus and the Akebiae Fructus has an anticancer effect and an insecticidal effect.

Hereinafter, a method of producing a composition for preventing hair loss and promoting hair growth according to the present invention by using natural materials as described above will be explained in detail.

Akebia Trifoliata of about 20˜40 g, Akebia Trifoliata root of about 20˜40 g, Akebiae Fructus of about 20˜40 g and Selaginella of about 20˜40 g may be put into purified water of about 1,500˜2,000 cc. At this time, it should be appreciated by one of ordinary skill that Akebia Trifoliata, Akebia Trifoliata root, and Akebiae Fructus may be added into purified water in finely sliced form. If the amount of each of these additive materials is less than 20 g, each of the materials will not be able to exhibit its effectiveness sufficiently. If the amount of each of these additive materials is more than 40 g, the time for preparing the base solution will be prolonged. As the amount of the additive materials increases, the amount of purified water also increases. Accordingly, it should be appreciated by one of ordinary skill that the above-mentioned range is suitable.

It should be noted that in the present invention, the use of all four substances such as Akebia Trifoliata, Akebia Trifoliata root, Akebiae Fructus and Selaginella is intended to utilize all of these active ingredients.

Akebia Trifoliata of about 20˜40 g, Akebia Trifoliata root of about 20˜40 g, Akebiae Fructus of about 20˜40 g and Selaginella of about 20˜40 g may be put into purified water and then this mixed solution is heated at the temperature of 100° C. for 30 minutes and then heating process is stopped and it is kept until the amount of the purified water is up to ⅔ of its original volume. Thereafter, an Akebia Quinata base solution may be obtained by cooling the mixed solution (=step (S1)).

Then, glutinous rice powders and corn flours may be put into the base solution obtained from the step (S1) and then it may be shaken to mix. Next, it may be heated at a predetermined temperature (=step (S2)).

In this step (S2), glutinous rice powders of about 20˜30 g, corn flours of about 20˜30 g may be put into the Akebia Quinata base solution of about 700˜1,000 cc. If the amount of each of glutinous rice powders and corn flours is too small, each of these materials will not be able to exhibit its effectiveness. If the amount of each of glutinous rice powders and corn flours is too large, the mixed solution is likely to be solidified. Accordingly, it should be appreciated by one of ordinary skill that the above-mentioned range is suitable.

The mixed solution may be continuously heated at a temperature of 800˜100° C. until the mixed solution becomes a gel-like state. The reason for performing the heating at this step (S2) is that it is to change physical properties of such additive materials containing starch into a water-soluble state. If the heating temperature is lower than 80° C., the heating time becomes longer. If the mixed solution is heated for about 1 to 10 minutes under a temperature range of 80 to 100° C., the solution becomes a gel-like state.

Next, the gel-like mixture resulted from the step (S2) may be filtered by using a mesh filter having a mesh size less than 300 mesh to remove debris (=step(S3). Next, the final product is packaged in a container after the disinfection process thereof has been completed.

### Embodiment 1

Akebia Trifoliata and Akebia Trifoliata root were obtained as raw materials and pre-sliced in a predetermined size and then they were either dried naturally or dried in a dryer. Likewise, Akebiae Fructus and Selaginella were obtained as raw materials and cut in a predetermined size and then they were either dried naturally or dried in a dryer. Then, Akebia Trifoliata of about 25 g, Akebia Trifoliata root of about 25 g, Akebiae Fructus of about 30 g and Selaginella of about 20 g were put into purified water of about 1,500 cc and then this mixed solution was heated at the temperature of 100° C. and then heating process was stopped and it was kept until the amount of the purified water is up to ⅔ of its original volume.

Thereafter, a base solution had been obtained by cooling the mixed solution. The volume of the base solution was about 980 cc.

Then, glutinous rice powders of about 20 g, corn flours of about 20 g were put into the base solution of about 700 cc and then it were shaken to mix. Next, it was heated at a temperature of 100° C. for about 7 minutes and then heating process was stopped and it was kept until the mixed solution becomes a gel-like state. Then, it was cooled in air and then filtered by using 300 mesh filter to remove debris. Finally, it was sterilized with ultraviolet rays and then it was placed in a sealed container and stored in a refrigerator.

### Embodiment 2

Akebia Trifoliata and Akebia Trifoliata root were obtained as raw materials and pre-sliced in a predetermined size and then they were either dried naturally or dried in a dryer. Likewise, Akebiae Fructus and Selaginella were obtained as raw materials and cut in a predetermined size and then they were either dried naturally or dried in a dryer. Then, Akebia Trifoliata of about 30 g, Akebia Trifoliata root of about 30 g, Akebiae Fructus of about 20 g and Selaginella of about 40 g were put into purified water of about 1,700 cc and then this mixed solution was heated at the temperature of 100° C. and then heating process was stopped and it was kept until the amount of the purified water is up to ⅔ of its original volume.

Thereafter, a base solution had been obtained by cooling the mixed solution. The volume of the base solution was about 1,100 cc.

Then, glutinous rice powders of about 30 g, corn flours of about 25 g were put into the base solution of about 1,100 cc and then it were shaken to mix. Next, it was heated at a temperature of 100° C. for about 10 minutes and then heating process was stopped and it was kept until the mixed solution becomes a gel-like state. Then, it was cooled in air and then filtered by using 300 mesh filter to remove debris. Finally, it was sterilized with ultraviolet rays and then it was placed in a sealed container and stored in a refrigerator.

### Embodiment 3

Embodiment 3 was performed by using the same method as in Embodiment 2. Akebia Trifoliata and Akebia Trifoliata root were obtained as raw materials and pre-sliced in a predetermined size and then they were either dried naturally or dried in a dryer. Likewise, Akebiae Fructus and Selaginella were obtained as raw materials and cut in a predetermined size and then they were either dried naturally or dried in a dryer. Then, Akebia Trifoliata of about 40 g, Akebia Trifoliata root of about 20 g, Akebiae Fructus of about 20 g and Selaginella of about 25 g were put into purified water of about 2,000 cc and then this mixed solution was heated at the temperature of 100° C. and then heating process was stopped and it was kept until the amount of the purified water is up to ⅔ of its original volume.

Thereafter, a base solution had been obtained by cooling the mixed solution. The volume of the base solution was about 1,300 cc.

Then, glutinous rice powders of about 25 g, corn flours of about 25 g were put into the base solution of about 1,300 cc and then it were shaken to mix. Next, it was heated at a temperature of 100° C. for about 10 minutes and then heating process was stopped and it was kept until the mixed solution becomes a gel-like state. Then, it was cooled in air and then filtered by using 300 mesh filter to remove debris. Finally, it was sterilized with ultraviolet rays and then it was placed in a sealed container and stored in a refrigerator.

### Embodiment 4

Embodiment 4 was performed by using the same method as in Embodiment 1. Akebia Trifoliata and Akebia Trifoliata root were obtained as raw materials and pre-sliced in a predetermined size and then they were either dried naturally or dried in a dryer. Likewise, Akebiae Fructus and Selaginella were obtained as raw materials and cut in a predetermined size and then they were either dried naturally or dried in a dryer. Then, Akebia Trifoliata of about 20 g, Akebia Trifoliata root of about 20 g, Akebiae Fructus of about 30 g and Selaginella of about 30 g were put into purified water of about 2,000 cc and then this mixed solution was heated at the temperature of 100° C. and then heating process was stopped and it was kept until the amount of the purified water is up to ⅔ of its original volume.

Thereafter, a base solution had been obtained by cooling the mixed solution. The volume of the base solution was about 1,000 cc.

Then, glutinous rice powders of about 25 g, corn flours of about 25 g were put into the base solution of about 1,000 cc and then it were shaken to mix. Next, it was heated at a temperature of 100° C. for about 7 minutes and then heating process was stopped and it was kept until the mixed solution becomes a gel-like state. Then, it was cooled in air and then filtered by using 300 mesh filter to remove debris. Finally, it was sterilized with ultraviolet rays and then it was placed in a sealed container and stored in a refrigerator.

### Embodiment 5

Akebia Trifoliata and Akebia Trifoliata root were obtained as raw materials and pre-sliced in a predetermined size and then they were either dried naturally or dried in a dryer. Likewise, Akebiae Fructus and Selaginella were obtained as raw materials and cut in a predetermined size and then they were either dried naturally or dried in a dryer. Then, Akebia Trifoliata of about 25 g, Akebia Trifoliata root of about 25 g, Akebiae Fructus of about 25 g and Selaginella of about 35 g were put into purified water of about 1,700 cc and then this mixed solution was heated at the temperature of 100° C. for 30 minutes and then heating process was stopped and it was kept until the amount of the purified water is up to ⅔ of its original volume.

Thereafter, a base solution had been obtained by cooling the mixed solution. The volume of the base solution was about 1,000 cc.

Then, glutinous rice powders of about 30 g, corn flours of about 30 g were put into the base solution of about 1,000 cc and then it were shaken to mix. Next, it was heated at a temperature of 100° C. for about 7 minutes and then heating process was stopped and it was kept until the mixed solution becomes a gel-like state. Then, it was cooled in air and then filtered by using 300 mesh filter to remove debris. Finally, it was sterilized with ultraviolet rays and then it was placed in a sealed container and stored in a refrigerator.

### Embodiment 6

Embodiment 6 was performed by using the same method as in Embodiment 5. Akebia Trifoliata and Akebia Trifoliata root were obtained as raw materials and pre-sliced in a predetermined size and then they were either dried naturally or dried in a dryer. Likewise, Akebiae Fructus and Selaginella were obtained as raw materials and cut in a predetermined size and then they were either dried naturally or dried in a dryer. Then, Akebia Trifoliata of about 23 g, Akebia Trifoliata root of about 24 g, Akebiae Fructus of about 20 g and Selaginella of about 27 g were put into purified water of about 1,500 cc and then this mixed solution was heated at the temperature of 100° C. for 30 minutes and then heating process was stopped and it was kept until the amount of the purified water is up to ⅔ of its original volume.

Thereafter, a base solution had been obtained by cooling the mixed solution. The volume of the base solution was about 950 cc.

Then, glutinous rice powders of about 25 g, corn flours of about 25 g were put into the base solution of about 950 cc and then it were shaken to mix. Next, it was heated at a temperature of 100° C. for about 7 minutes and then heating process was stopped and it was kept until the mixed solution becomes a gel-like state. Then, it was cooled in air and then filtered by using 300 mesh filter to remove debris. Finally, it was sterilized with ultraviolet rays and then it was placed in a sealed container and stored in a refrigerator.

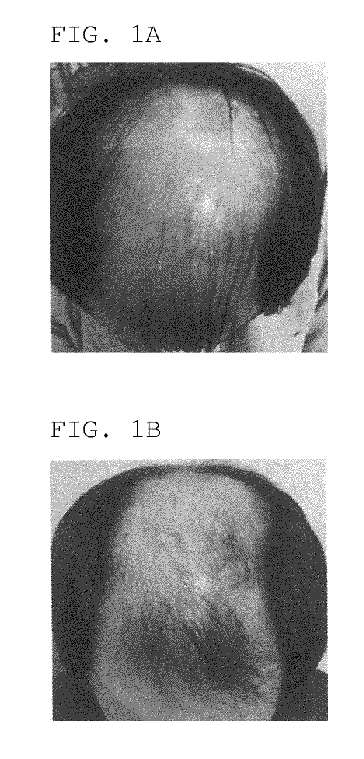

Herein below experimental examples according to the present invention will be explained with reference to FIGS. 1A to 6B.

### Experimental Example 1

FIGS. 1A and 1B are photographs taken before and after an experiment with a composition for preventing hair loss and promoting hair growth according to a preferred first embodiment of the present invention. FIG. 1A shows a hair condition of a test subject in the control group taken before the experiment with the composition. The test subject sprayed the composition on his hair twice a day and then rinsed it with water after about 50 minutes had elapsed. He used it for 10 months in this way. The composition was stored frozen before use and it was refrigerated during use. FIG. 1B shows a hair condition of the test subject in the control group taken after the experiment with the composition. As shown in FIG. 1B, it can be seen that a lot of hairs were formed by virtue of using the composition for 10 months.

### Experimental Example 2

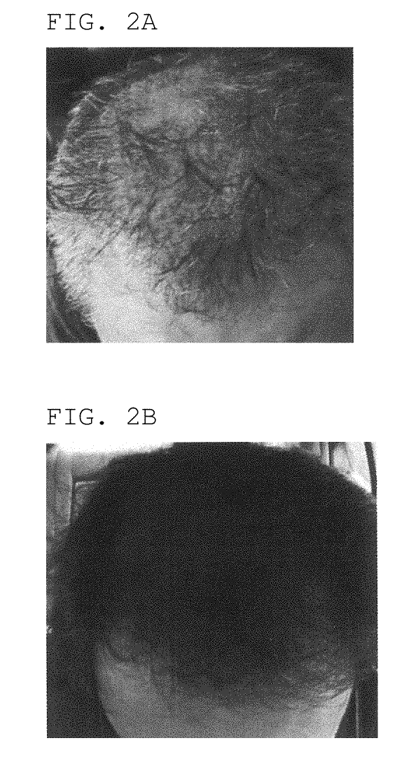

FIGS. 2A and 2B are photographs taken before and after an experiment with a composition for preventing hair loss and promoting hair growth according to a preferred second embodiment of the present invention. FIG. 2A shows a hair condition of a test subject in the control group taken before the experiment with the composition. The test subject sprayed the composition on his hair three times a day and then rinsed it with water after about 50 minutes had elapsed. He used it for 7 months in this way. The composition was stored frozen before use and it was refrigerated during use. FIG. 2B shows a hair condition of the test subject in the control group taken after the experiment with the composition. As shown in FIG. 2B, it can be seen that a lot of hairs were formed by virtue of using the composition for 7 months.

### Experimental Example 3

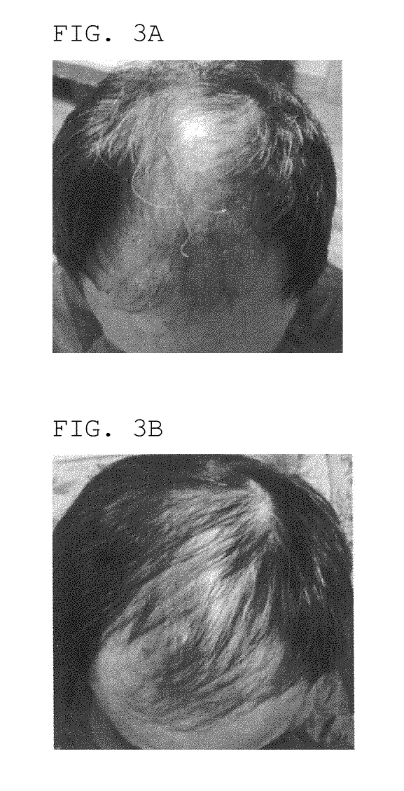

FIGS. 3A and 3B are photographs taken before and after an experiment with a composition for preventing hair loss and promoting hair growth according to a preferred third embodiment of the present invention. FIG. 3A shows a hair condition of a test subject in the control group taken before the experiment with the composition. The test subject sprayed the composition on his hair twice a day and then rinsed it with water after about 50 minutes had elapsed. He used it for 5 months in this way. The composition was stored frozen before use and it was refrigerated during use. FIG. 3B shows a hair condition of the test subject in the control group taken after the experiment with the composition. As shown in FIG. 3B, it can be seen that a lot of hairs were formed by virtue of using the composition for 5 months.

### Experimental Example 4

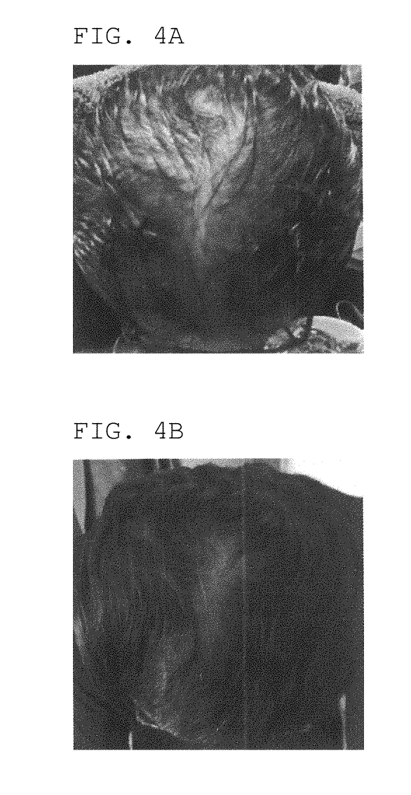

FIGS. 4A and 4B are photographs taken before and after an experiment with a composition for preventing hair loss and promoting hair growth according to a preferred fourth embodiment of the present invention. FIG. 4A shows a hair condition of a test subject in the control group taken before the experiment with the composition. The test subject sprayed the composition on his hair three times a day and then rinsed it with water after about 50 minutes had elapsed. He used it for 4 months in this way. The composition was stored frozen before use and it was refrigerated during use. FIG. 4B shows a hair condition of the test subject in the control group taken after the experiment with the composition. As shown in FIG. 4B, it can be seen that a lot of hairs were formed by virtue of using the composition for 4 months.

### Experimental Example 5

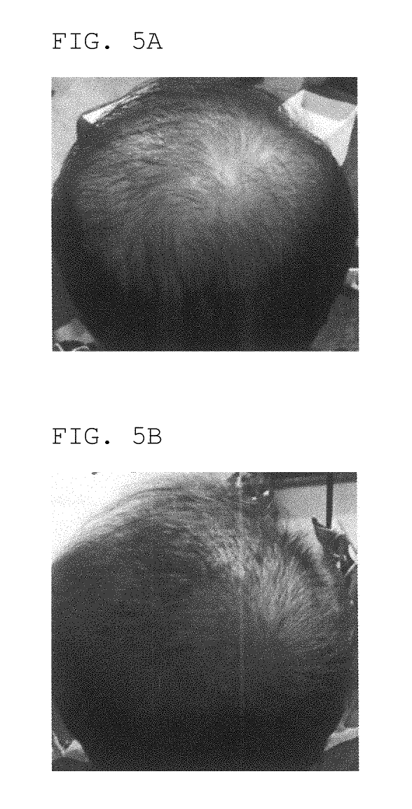

FIGS. 5A and 5B are photographs taken before and after an experiment with a composition for preventing hair loss and promoting hair growth according to a preferred fifth embodiment of the present invention. FIG. 5A shows a hair condition of a test subject in the control group taken before the experiment with the composition. The test subject sprayed the composition on his hair three times a day and then rinsed it with water after about 50 minutes had elapsed. He used it for 3 months in this way. The composition was stored frozen before use and it was refrigerated during use. FIG. 5B shows a hair condition of the test subject in the control group taken after the experiment with the composition. As shown in FIG. 2B, it can be seen that a lot of hairs were formed by virtue of using the composition for 3 months.

### Experimental Example 6

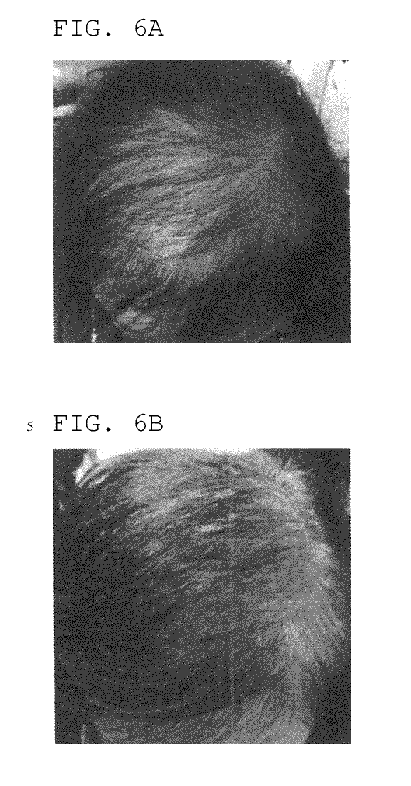

FIGS. 6A and 6B are photographs taken before and after an experiment with a composition for preventing hair loss and promoting hair growth according to a preferred sixth embodiment of the present invention. FIG. 6A shows a hair condition of a test subject in the control group taken before the experiment with the composition. The test subject sprayed the composition on his hair three times a day and then rinsed it with water after about 50 minutes had elapsed. He used it for 8 months in this way. The composition was stored frozen before use and it was refrigerated during use. FIG. 6B shows a hair condition of the test subject in the control group taken after the experiment with the composition. As shown in FIG. 6B, it can be seen that a lot of hairs were formed by virtue of using the composition for 8 months.

As described above, it can be seen that the composition according to the present invention can be used not only for hair loss prevention and hair growth but also as a raw material for cosmetics or as an external preparation for scalp.

Although the invention is described with respect to specific embodiments, the principles of the invention, as defined by the claims appended herein, can obviously be applied beyond the specific embodiments of the invention described herein. The foregoing description of preferred embodiments of the present invention has been presented for the purposes of illustration and description. It is not intended to be exhaustive or to limit the invention to the precise form disclosed. Modifications and variations are possible in light of the above teachings, or may be acquired from practice of the invention. The embodiments were chosen and described in order to explain the principles of the invention and its practical application to enable one skilled in the art to utilize the invention in various embodiments and with various modifications as are suited to the particular use contemplated. It is intended that the scope of the invention be defined by the claims appended hereto, and their equivalents.

1. A method of producing a composition for preventing hair loss and promoting hair growth, the method comprising the following steps:
(S1): putting an Akebia Quinata and a Selaginella into purified water and then heating the resulting solution so as to make an Akebia Quinata base solution;
(S2): adding glutinous rice powders and corn flours to the Akebia Quinata base solution resulted from the step (S1), and then mixing and heating them so as to prepare a gel-like mixture; and
(S3) cooling, filtering and sterilizing the gel-like mixture resulted from the step (S2).

2. The method as claimed in claim 1, wherein in the step (S1), the Akebia Quinata comprises at least one material selected from the group consisting of Akebia Trifoliata that is a stem of the Akebia Quinata, Akebia Trifoliata root that is a root of the Akebia Quinata, and Akebiae Fructus that is a fruit of the Akebia Quinata, in which the sum of the Akebia Quinata and the Selaginella to be used may be 90 to 150 g based on the purified water of 1,500˜2,000 cc.

3. The method as claimed in claim 1, wherein in the step (S1), after putting the Akebia Quinata and the Selaginella into the purified water, the resulting solution is heated for 30 minutes at 100° C. and then heating process is stopped and then it is kept until the amount of the purified water is up to ⅔ of its original volume.

4. The method as claimed in claim 1, wherein in the step (S2), the sum of the glutinous rice powder and the corn flour to be used may be 40 to 60 g based on the Akebia Quinata base solution of 900˜1,000 cc.

5. The method as claimed in claim 1, wherein in the step (S3), the filtering process is performed by using a 300 mesh filter.
